(19) United States
(12) Patent Application Publication
(10) Pub. No.: US 2011/0098571 A1
(43) Pub. Date: Apr. 28, 2011

(54) SCAN LINE DISPLAY APPARATUS AND METHOD

(76) Inventors: Andrew John Medlin, Thebarton (AU); Andrew John Paul Niemiec, Thebarton (AU)

(21) Appl. No.: 12/674,007

(22) PCT Filed: Aug. 29, 2008
(86) PCT No.: PCT/AU2008/001277

§ 371 Date: Feb. 18, 2010

(30) Foreign Application Priority Data
Aug. 31, 2007 (AU) 2007904743

(51) Int. Cl. A61B 8/14

(52) U.S. Cl. 600/445

(57) ABSTRACT

A method for forming an image of a target from echo return data in an ultrasound system by receiving a set of scanlines from an ultrasound transducer and applying a transform to map the scanlines to a plane of best fit, then mapping and interpolating the transformed data to a raster image and displaying the resultant image.

### TECHNICAL FIELD

The present invention relates to an improved method and apparatus for displaying scanlines from an ultrasound probe on a video display. The method has particular application to the field of hand-held ultrasound equipment.

### BACKGROUND ART

Ultrasound was first investigated as a medical diagnostic imaging tool in the 1940's. This was based on the use of A-mode (amplitude mode) ultrasound, which is a form of echo ranging. This simply gives a plot of returned echo intensity against time, which, by knowing the speed of sound in the target media, gives the distance of the features returning the echo from the transducer. In order to obtain valid information from such a scanline it is necessary to hold the direction of the transmitted ultrasound beam constant and known.

In order to provide an imaging system, it is necessary to insonify a larger area, at least a two dimensional slice of the target. It is also necessary to receive returned echoes from this area and to display this information in correct spatial relationship.

Since the only information received by an ultrasound transducer is echo intensity over time, spatial information can most easily be added by knowing the direction from which the echo was received. This means knowing the position and orientation of the transducer at all times and this was most easily achieved by controlling the movement of the transducer.

This led to B-mode (brightness mode) scanning, where the ultrasound output is pulsed and the transducer is mechanically scanned over the target. The transducer detects the echo from each pulse as intensity versus time, called a scanline. The scanlines are displayed with brightness being proportional to echo intensity, thus forming an image.

Articulated arm scanners, also known as static mode scanners, connect the ultrasonic transducer to a moveable arm, with movement of the arm mechanically measured using potentiometers. The articulated arm also ensures that the degree of freedom of movement of the transducer is limited to a defined plane. This allowed the position of the transducer to be known with considerable accuracy, thus allowing the scanlines recorded by the transducer to be accurately located in space relative to each other for display.

The static mode scanners were large cumbersome devices, and the techniques used are not readily suited to a handheld ultrasound system.

A B-mode scanning system may be constructed using a mechanically mounted rotating transducer. Motor driven transducers removed the need for precise knowledge of the position of the transducer housing, since the operator needed only to hold the transducer housing still and the motor would sweep the transducer rapidly to produce a scan arc. This results in an evenly distributed set of scanlines, in a single plane, whose spatial relationship is known because the sweep characteristics are known.

The motor driving circuitry, adds size, power consumption, complexity and cost to the device. Additionally, the motor itself and associated moving parts reduce the reliability of the device.

A solution to these problems is boon found in electronic beam steering transducers consisting of a number of electronic crystals where the transmitting pulse can be delayed in sequence to each crystal and effect an electronic means to steer the ultrasound beam. Nearly all modern medical ultrasound equipment uses an array of ultrasonic crystals in the transducer. The early designs used at least 64 crystals, with modern designs sometimes using up to a thousand crystals or more.

Electronic beam steering removes the need for a motor to produce real time images. The scanlines resulting from the use of an array transducer are contained within a defined plane, or in the case of 2-D arrays within a defined series of planes. The scanlines may therefore be readily mapped onto a flat screen for display.

However, the cost of producing transducers with arrays of crystals is high. There is also a high cost in providing the control and processing circuitry, with a separate channel being required for each crystal. The transducers are usually manually manufactured, with the channels requiring excellent channel to channel matching and low cross-talk. The power consumption for electronic systems is also high, and is generally proportional to the number of channels being simultaneously operational.

Much of the prior art in ultrasound technology is directed to improving the performance of ultrasound systems enabling them to be used for an ever increasing range of diagnostic applications. The result has seen significant advances in ultrasound systems with transducers using over increasing numbers of crystals, and host systems with ever increasing processing power. The result has seen systems with 3D and real-time 3D (or 4D) capability.

These high cost, high power consumption devices are unsuitable for broad point-of-care application outside of specialist sonography facilities. In particular, these systems are unsuitable for application to hand-held devices. Providing useful images from simpler transducer arrangements, which are suitable for hand-held use, within the prior art is difficult in part because of the difficulty of providing a uniformly distributed set of scanlines in a single scan plane.

### DISCLOSURE OF THE INVENTION

A handheld ultrasound device of low cost may be implemented which isonifles a region of interest by sweeping a single or a small number of ultrasound “beams” over the region. The sweeping may be by manual movement of a probe unit including an ultrasound transducer. The direction of the probe unit and hence the beam may be determined by position measurement means such as a gyroscope. Position data and the echoes received from the emitted beams are combined to give a series of scanlines covering the region of interest it is necessary to efficiently process this scanline data set to produce an image for display to a user. For a 2D display, this image will appear as a plane through the region of interest.

Therefore, in one form of this invention although this may not necessarily be the only or indeed the broadest form of this there is proposed a method for forming an image of a target from echo return data in an ultrasound system including the steps of

receiving a plurality of scanlines from an ultrasound transducer
applying a transform to map the scanlines to a plane of best fit mapping and interpolating the transformed data to a raster image displaying the resultant image.

Various mathematical methods may be used to calculate the transform to the plane of best fit.

In preference said transform is calculated by principal component analysis.

In preference principal component analysis is applied only to selected data points of a scanline.

In preference the selected data points are the first and last points of the scanline.

The planar scanline dataset must now be displayed as a raster image. This means that the data points of the scanlines, which are distributed arbitrarily across the plane of best fit must be mapped to a regular array of display pixels.

The efficiency of this mapping process is affected by the choice of co-ordinate system used to describe and store the pixel array. An efficient method of performing this mapping is proposed.

In preference the mapping and interpolation is undertaken using a method we describe as pixel row-wise interpolation, which incorporates the use of a coordinate system we have called the pixel-scanline co-ordinate system.

In a further form, the invention may be said to ile in an apparatus for forming an image in an ultrasound system including a probe unit having at least one transducer adapted to emit and receive a single ultrasound scanline

the probe unit including a sensor adapted to sense at least one of the probe unit's position and orientation
a data processing and display unit adapted to process and display the processed scanline data as an ultrasound image,
wherein in use the instantaneous output of said sensor is combined with corresponding transducer output data to form a scanline
the scanline data is transmitted to a data processing and display unit adapted to process and display a plurality of scanlines, the processing of the scanlines including applying a transform to map the scanlines to a plane of best fit;
mapping and interpolating the transformed data to a raster image.

### BRIEF DESCRIPTION OF THE DRAWINGS

FIG. 1 shows an ultrasonic scan system including an embodiment of the invention;

FIG. 2 shows a probe unit with a gyroscope as the position/orientation sensor;

FIG. 3 shows a graphical representation of an ultrasound scan data set;

FIG. 4 shows two possible relationships between scan geometry and screen display;

FIG. 5 shows an ultrasound scan space, with the pixel grid of a pixel buffer overlaid upon it.

FIG. 6 shows a partial ultrasound scan space, with the pixel grid of a pixel buffer overlaid upon it, illustrating scanline/rowline intersection;

FIG. 7 shows a partial ultrasound scan space, with the pixel grid of a pixel buffer overlaid upon it, illustrating scanline and intersection point ordering;

FIG. 8 shows the selection of a scan data point as a pixel value.

FIG. 9 illustrates a co-ordinate system for a scanline.

### BEST MODE FOR CARRYING OUT THE INVENTION

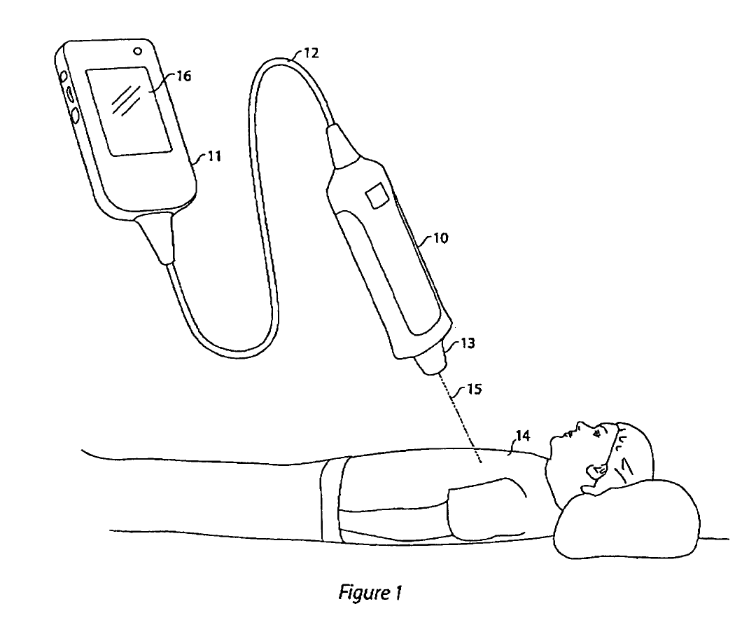

Referring now to FIG. 1, there is illustrated an ultrasonic scan system according to an embodiment of the invention. There is a hand held ultrasonic probe unit 10, a display and processing unit (DPU) 11 including a display screen 18 and a microprocessor. There is a cable 12 connecting the probe unit 10 to the DPU 11.

The probe unit 10 includes an ultrasonic transducer 13 adapted to transmit pulsed ultrasonic signals into a target body 14 and to receive returned echoes from the target body 14.

In this embodiment, the transducer is capable of producing a single scanline 15, at a fixed orientation to the probe unit.

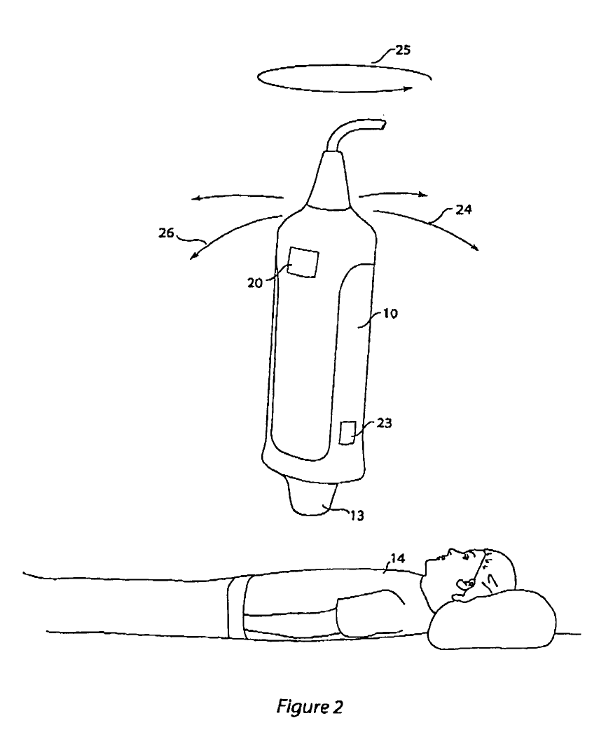

As shown in FIG. 2, the probe unit further includes an orientation sensor 20 capable of sensing orientation or relative orientation about one or more axes of the probe unit. Thus, in general, the sensor is able to sense rotation about any or all of the axes of the probe unit, as indicated by rotation arrows 24, 25, 26.

The sensor may be implemented in any convenient form. In an embodiment the sensor consists of three orthogonally mounted gyroscopes. In further embodiments the sensor may consist of two gyroscopes, which would provide information about rotation about only two axes, or a single gyroscope providing information about rotation about only a single axis.

In the currently preferred embodiment, the sensor is an inertial sensor in the form of a gyroscope 20 positioned to measure rotation about the z axis of the probe unit, as shown in FIG. 2. It can be seen that the direction information for a scanline will include information for only one degree of freedom.

In further embodiments, the position and/or orientation sensor may be any combination of gyroscopes and accelerometers mounted in relative position to one another so as to give information about the linear and angular displacement of the probe unit. Full relative position data for the probe unit can be obtained with three orthogonally mounted accelerometers and three orthogonally mounted gyroscopes. This arrangement provides measurement of displacement in any direction and rotation about any axis. This allows for direction information for a scanline to be given for all six degrees of freedom.

In embodiments, direction information for scanlines may be available for any number of possible degrees of freedom.

In another embodiment, the position and/or orientation sensing means is an electromagnetic spatial positioning system of the type requiring a fixed positioning transmitter separate from the probe unit, which transmits electromagnetic signals which are received by a receiver on the probe unit, the receiver providing information as to the position and orientation of the probe in the field of the transmitter.

The position and orientation means may be any suitable system or combination of systems which yields sufficient position information to form a useful image from the received scanlines. Optical positioning systems employing LED's and photodetectors may be used. This has the disadvantage of requiring line of sight access to the probe unit at all times.

Acoustic location systems may also be used combining a sound source on the probe with acoustic sensors at known points.

Visual tracking systems using a camera to observe the movement of the probe and translate this into tracking data could also be used. This also has the disadvantage of requiring line of sight access to the probe unit at all times.

All of these systems are less preferred in that they require apparatus which is in some way fixed in position, making them less suitable for use with a hand held ultrasound system.

In use, probe unit electronics apply an electrical pulse to the transducer 13. The transducer produces a scan ultrasonic pulse in response to each electrical pulse. This scan pulse travels into the body and is reflected from the features of the body to be imaged as an ultrasonic echo signal. This echo signal is received by the transducer and converted into an electrical receive signal.

At the same time, data is received from the position and/or orientation sensor. In the preferred embodiment, this is the angular change in the position of the probe unit since a selected preceding transducer pulse, usually the first pulse of a scan.

This is combined with the response signal to give a scanline. A scanline is a dataset which comprises a sequential series of intensity values of the response signal combined with position information. A scan dataset is a plurality of sequentially received scanlines.

In general, the position information in a scanline is the position in space of the probe unit and its angle of rotation. Each of these may be with respect to some absolute position, or simply relative to any previous scanline, in particular the immediately preceding scanline, in the scan data set.

The scan data set is built up by a user moving the probe unit in a defined way to scan the target body. The probe electronics continue to provide the electrical pulses to the transducer and each pulse results in a scanline.

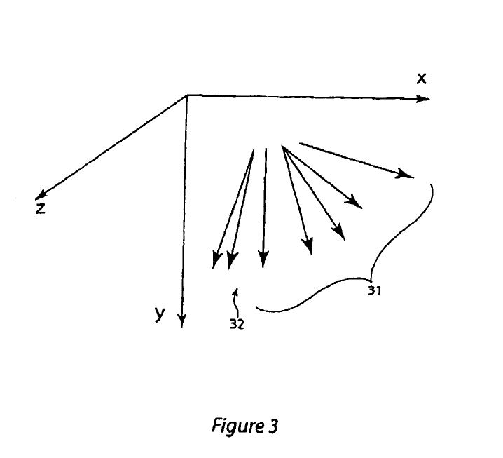

The result is a scan data set, as illustrated in FIG. 3. The scan data set may be seen to consist of a series of scanlines 31, wherein an individual scanline 32 has an origin, a direction, and a depth. Taken together, these constitute the echo data for some geometric region in the target body. In general the origin and direction and depth of each scanline may be different. In a preferred embodiment and method of use, the origins will be closely grouped, the depths will be the same while the directions will vary to give an approximately fan shaped data set.

The defined way in which the user moves the probe unit is defined to give suitable results having regard to the information available from the position and/or orientation sensor. In an embodiment where the sensor provides only orientation data, translational movement of the probe is avoided as much as possible. In an embodiment where the position and/or orientation sensor provides information only of rotation about a single axis, rotation about any other axis is also avoided as much as possible.

The scan data set is passed to the display and processing unit (DPU) 11 which includes a microcontroller and a display screen 16.

The data is processed by the DPU to produce a 2D image for display on the display screen.

The scanlines may be seen as existing in an arbitrary co-ordinate system, which we have called “capture space”. The position data component of the general scan data set may be seen as being in 3D Cartesian coordinates in capture space, with the origin of each scan line represented as a position vector and the direction as a unit vector in 3D space.

In the general case, the scan lines will not share a common plane, nor a common origin. In order to display the data it is necessary to transform the scan data set to “scan space”.

We define “scan space” as a Cartesian coordinate system optimally suited to the scan data set and oriented appropriately for ease of mapping and interpolation to a raster image. This may be thought of as a “plane of best fit”, the single plane which best characterises the 3D scan data in 2D, combined with an “origin of best fit”.

For a preferred embodiment, with the position and/or orientation sensor being a single gyroscope providing angular information about only one axis, the position vector of each scanline has the same value, and the direction vectors of each scanline are therefore defined to be co-planar, even though the actual movement of the probe in non-sensed directions would mean that this would cause a small error. The method of use, described above, ensures that the error caused by actual changes in the non-sensed parameters is sufficiently small to ensure that the error in the resultant image display is negligible.

In further embodiments where the position and orientation sensor provides other and further information, modes of use may be employed which require information about movement in other degrees of freedom to ensure accurate image presentation, in the case of embodiments employing absolute position and orientation determination, or a full set of inertial sensors, information on movement in all degrees of freedom is available.

Where such full information is collected, the probe unit may be rotated in an arbitrary direction, and/or moved translationally across the surface of the subject body.

The selection of the transform to scan space adheres to a number of user expectations in order to facilitate the interpretation of the image.

It is necessary to choose a forward direction for scan apace which will correspond to the vertical centerline of the screen display.

Conventionally the scan plane lies in the x-y plane, with the z-position being a measure of error from the optimal plane and the y-direction nominally being the scan forwards direction.

The transformation to scan space does not sense reverse the data. This ensures that the left-right orientation of the probe unit is always transformed to the display screen in the same way from scan to scan. The transform does not contain any reflection component about any plane. The transformation is purely rotational plus translational.

It is necessary that it be possible to determine a nominal forwards direction for scan space. Further, when the data set comes to be rendered into a display buffer for display, it is preferable that no scan line be parallel to a pixel row of that display buffer. In the preferred embodiment, both of these are facilitated by ensuring that the raw scan data spans less than 180 degrees of probe rotation.

Mathematically, transforms between co-ordinate systems can be represented by a four element by four element transform matrix. The transform from capture space to scan space is such a transform. Software in the DPU now calculates and applies this transform.

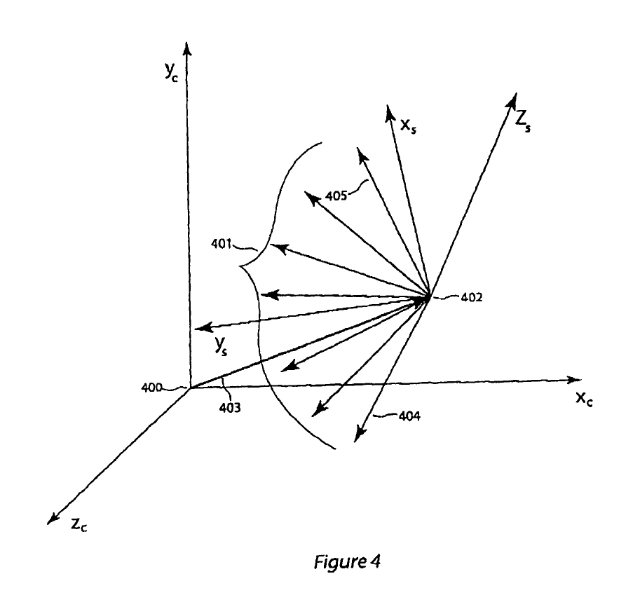

The nature of the transform may be understood by reference to FIG. 4. There are shown the scanlines 401 of a scan taken as previously described. These are shown with reference to an arbitrary capture space co-ordinate system, with origin 400 and co-ordinate axes \hat{x}_{c},ŷ\hat{y_{c}},\hat{z}_{c}. This is to be transformed to an optimal display co-ordinate system, scan space, with origin 402 and co-ordinate axes \hat{x}ₛ,ŷ\hat{yₛ},\hat{z}ₛ.

Scan space can be described in terms of capture space by the unit vectors
[\hat{x}ₛ,ŷ\hat{yₛ},\hat{z}ₛ]

The position of the origin 402 of scan space is represented by the vector P 403.

Thus the principles of vector mathematics yield the transform T from scan space to capture space as:

$$T = [ \begin{matrix}{{x}_{s}}^{⋒} & {{y}_{s}}^{⋒} & {{z}_{s}}^{⋒} & \overline{P} \\ 0 & 0 & 0 & 1\end{matrix} ]$$

The first step in determining T is to determine ŷₛ. This can be determined by following the conventions of ultrasound image display as described previously. The y direction of scan space is the scan forwards direction and it is desirable that the scan when displayed should fit centred on the display screen.

The orientation of each of the scanlines is examined to determine the extreme scanlines 404,405. These will not necessarily be first and last scanlines received. The direction that bisects the angle between the two extreme scanlines is taken as the unit vector ŷₛ.

With ŷₛ determined it is now possible to determine the unit vectors \hat{x}ₛ, and \hat{z}ₛ. This is equivalent to determining the “plane of best fit” for the scanlines. Principal component analysis (PCA) allows one to take a set of sample points in multiple dimensions and analyse it in a systematic way to produce a new basis in which to represent the data. It also provides a measure of how strongly the sample points relate to each basis component.

The result is a set of orthogonal unit vectors that comprise a transformation from the original, arbitrary, Cartesian coordinate system of the incoming spaclines which we have called capture space to a new coordinate system in which the transformed scanlines ile as close as possible to a statistically averaged x-y scan plane, a “plane of best fit” which we have called scan space.

PCA determines principal components in the data set by computing eigenvectors of a covariance matrix. These eigenvectors are the unit vectors defining the x and z axes of scan space. The magnitude of the eigenvalue corresponding to each eigenvector indicates how strongly the data correlates in that basis direction.

Mathematically, given a set of N points (xₗ,zₗ), l=1 . . . N, then the mean is:

$$( \overline{x} , \overline{z} ) = \frac{1}{N} ( {\sum }_{i = 1}^{N} {x}_{i} , {\sum }_{i = 1}^{N} {z}_{i} )$$

The statistical variances are:

$$\begin{matrix}{S}_{xx} = \frac{1}{N - 1} {\sum }_{i = 1}^{N} {( {x}_{i} - \overline{x} )}^{2} \\ {S}_{xx} = \frac{1}{N - 1} {\sum }_{i = 1}^{N} {( {z}_{i} - \overline{z} )}^{2} \\ {S}_{xx} = \frac{1}{N - 1} {\sum }_{i = 1}^{N} {( {x}_{i} - \overline{x} )}^{2} {( {z}_{i} - \overline{z} )}^{2}\end{matrix}$$

And the covariance matrix is:

$$C = ( \begin{matrix}{S}_{xx} & {S}_{xx} \\ {S}_{xx} & \text{?}\end{matrix} )$$

$$\text{?} \text{indicates text missing or illegible when filed}$$

Principal component analysis is undertaken in the scan space frame of reference. The unit vector ŷ_{z} has already been determined, it is necessary to determine \hat{x}_{z} and \hat{z}ₛ. In order to do this, an initial estimate of the unit vector \hat{z}ₛ is made, which also determines an estimate for \hat{x}_{z}. The choice of estimate is not critical.

A useful estimate for \hat{z}ₛ is a unit vector in the direction of the vector cross product of the two extreme scanlines 404, 405.

The scanlines are then transformed into this estimated scan space frame of reference. PCA is applied. The resultant primary eigenvector is the final value for \hat{x}_{z}, and the secondary eigenvector is \hat{z}ₛ.

The final step is to determine P. This can be determined as any point on the x [symbol] -y [symbol] plane where the average value of z [symbol] in the scan dataset is zero. Once this is determined, the transform T is known.

Once T is known, the capture space co-ordinates are transformed to scan space by multiplying their position and direction vectors by T⁻¹

Following this step, the square of each z [symbol] -coordinate is equal to the variance at that point, which can be used to measure scan quality. The lower the variance, the more closely the scanlines in capture space lie to the plane of best fit, and hence the less the displayed image has been distorted by the process of bringing it to 2D.

For display purposes, the z-coordinate values are now ignored, which has the effect of projecting the scanlines onto the scan, plane.

In principle PCA needs to be applied to all of the scan data points in the field of view. Since the number of scan points is the product of the number of scanlines and the number of points per scanline, this may be a very heavy computational load, ill suited to the modest computational capabilities of a hand-held device.

It has been discovered that applying PCA to only the first and last points on a scanline yields a transform which for practical purposes is the same as that yielded by the full analysis. Using this modified algorithm gives a large saving in computational effort, with negligible increased error in the final display.

The result of transforming the scanlines to scan space is a scan space data set.

It is now necessary to map scan space to the available display hardware. In an embodiment, this is the display screen 18. A minimal axis aligned bounding box is defined that is the minimum sized rectangle which will encompass all scanlines. It is defined as a rectangle with vertices:
(xₛ_{ₘᵢₙ},yₛ_{ₘᵢₙ}),(xₛ_{ₘₐₓ},yₛ_{ₘₐₓ})

A process we have called pixel row-wise scan interpolation is now applied to the scan space data in order to provide data suitable for display. Pixel row-wise scan interpolation is a general method for rendering unordered scan line data, where the order of the scan lines does not need to be pixel order. The scan data points may have an arbitrary spatial density, which need not be uniform.

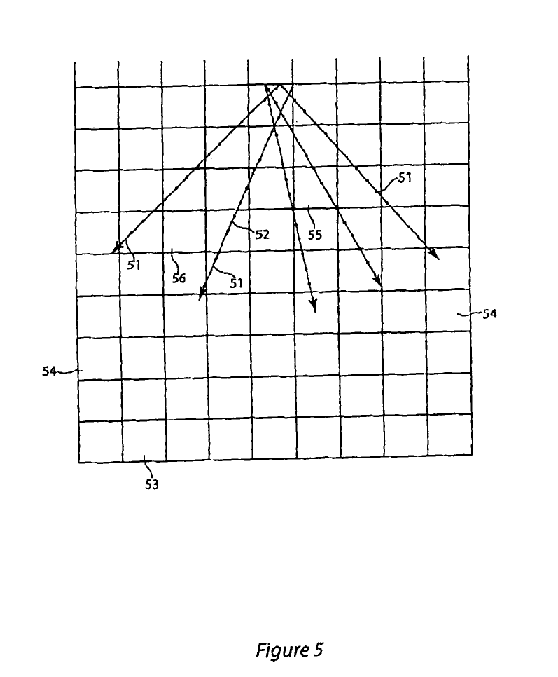

As shown in FIG. 5, the scan space data is a series of scanlines 51 in a common plane. Each scanline consists of a number of data points 52. In the case of an ultrasound scan these are intensity of reflection values. For the purposes of display these are brightness values.

FIG. 5 also shows a pixel grid superimposed on the data. This is a pixel buffer having known pixel dimensions and the same aspect ratio as the bounding box. As can be seen, a pixel buffer is a regular grid 53 of individual pixels 54. Each pixel can have only one brightness value. It can be seen that there are pixels 55 which are associated with more than one scan data point and other pixels 56, which are associated with none. Pixel row-wise scan interpolation is applied to produce a data set with one and only one brightness value associated with each pixel.

Pixel row-wise interpolation begins by intersecting the scan lines with the pixel buffer one pixel row at a time. The algorithm is comprised of straightforward 2D geometrical calculations that are highly data coherent and therefore well suited to a hand-held processing device in which computational resources may be limited.

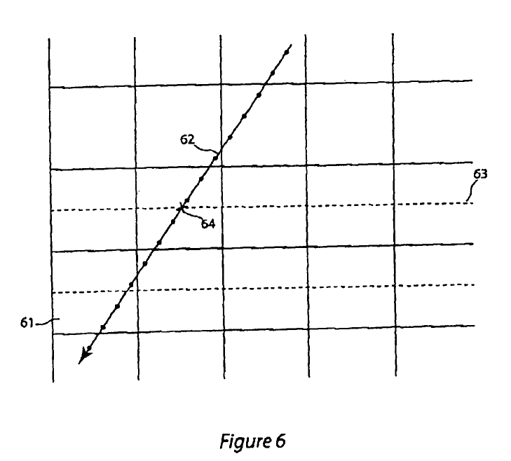

Looking at FIG. 6, there is a pixel row 61 and a scanline 62. For ease of description, rows are shown horizontally, and we will refer to pixel height and width, however it will be obvious that this does not restrict the orientation at which a final image is displayed, nor is there any required relationship between the direction and angle of the scanlines and the pixel grid.

We define a rowline 63 as the midline of the pixel row. There is one intersection point 64 between the rowline and the scanline. This intersection point is at a defined point with respect to the height of the pixel, but will be at an arbitrary distance across the width of the pixel. Accordingly, the intersection position is calculated in fraction of pixel co-ordinates. The intersection is described and stored in a co-ordinate system we have called scanline-pixel co-ordinates, as further described below.

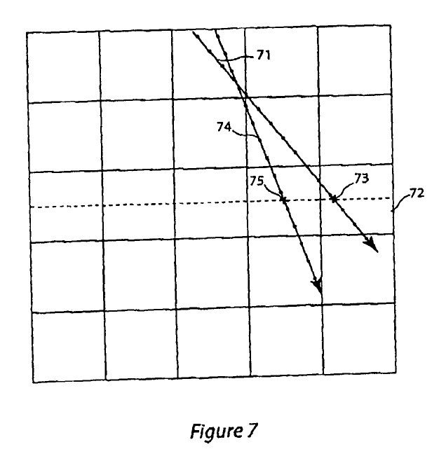

Each of these intersection points is calculated for a given row. This gives an array of values sorted in the order of the received scanlines. This may not be the order of the column of the pixel grid. As shown in FIG. 7, scanline 71 intersects rowline 72 at intersect point 73, while scanline 74 intersects rowline 72 at intersect point 75. Scanline 71 precedes scanline 74 in the scan data set, but intersection point 73 is later in pixel column order. This can occur because the ultrasound probe unit, being hand scanned, may briefly wobble in a direction against the predominant direction of rotation. It is also possible for linear displacement of the transducer during a scan to cause scanlines to overlap.

The calculated intersection points are now sorted into pixel column order, and order within each pixel in the case where more than one intersection point occurs within a pixel.

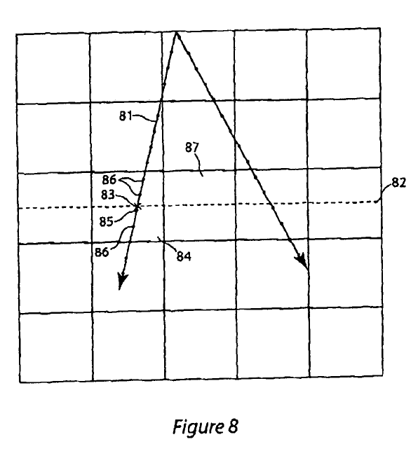

The brightness value which is assigned to each pixel is chosen as that of the discrete data point which is closest to the intersection point. This is shown on FIG. 8. Scanline 81 intersects rowline 82 at intersect point 83 in pixel 84. Scan data point 85 is closest to the intersection point and becomes the value for pixel 84. Scan data points 86, in the same pixel, are ignored and do not contribute to the displayed image.

There may be more than one intersect point in a pixel. In this case, the pixel value is the mean of the value of the data points which are closest to each of the intersect paints.

Also in FIG. 8, there are shown pixels 87 which are “holes”, that is they do not have a scanline intersect. In order to display a smooth image, these holes must be filled with values which are consistent with the filled pixels around them.

This is done by interpolation between pixels having defined values. Where linear interpolation is employed, the brightness value for the holes is defined such that there is a constant increment between the brightness values of the holes and adjacent pixels.

Other interpolation formulae may be used to fill in the values for the holes. The interpolation of the preferred embodiment is linear but quadratic, cubic or other higher order interpolations may be used.

Having calculated one row of the pixel grid the remainder of the grid is now calculated. Calculation of the intersection points between the scanlines and the mid-lines of the pixel rows is made surprisingly straightforward by the method of the invention.

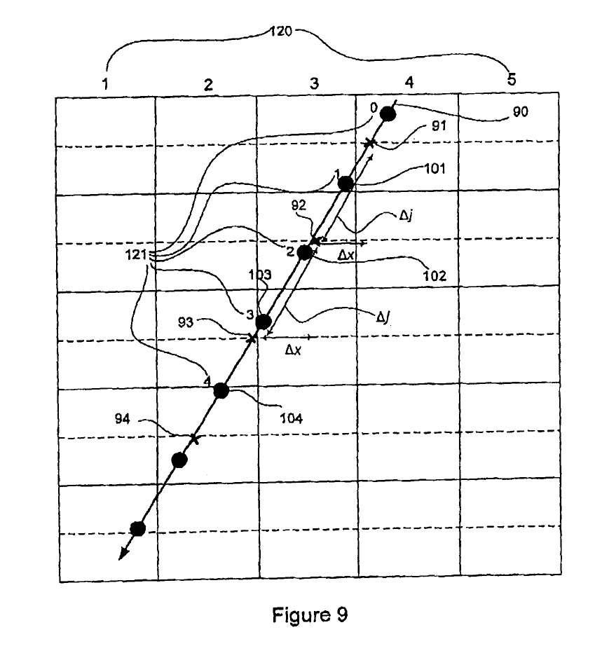

FIG. 9 shows a pixel grid, with each pixel having a row index number. Shown on the grid is a scanline 90, having scan data points 101,102,103,104. There are intersection points 91,92,93,94 between the scan line and the grid midlines. The scanline 90 is one scan line from a data set of N scanlines which may be identified as being denoted k=0, 1, 2, . . . N−1. Each scanline consists of an array of J data points which may be identified as being denoted zⱼ for j=0, 1, 2, . . . J−1

Since the scan data points are equidistant, the position of each data point along the scanline may be described by an index number 121.

Accordingly, the location of each intersection point can be described and stored in a scanline-pixel co-ordinate system, with co-ordinates Cₖ=(xₖ,jₖ), where xₖ is a fractional pixel index and jₖ is a fractional scan data point index. For example, the co-ordinates of intersection point 91 would be (3.1,0.6).

There is a vector [symbol] ₖ for each pixel row which describes the intersection points of all the scanlines for that pixel row.

After the first row of pixels, subsequent intersection points are determined simply by adding a constant offset to the fractional pixel and scan line coordinates.

Referring to FIG. 9, it can be seen that, having established intersection point 91, the location of intersection point 92 in the scanline-pixel co-ordinate system can be found by adding Δx to the pixel co-ordinate and Δj to the scanline co-ordinate. Intersection point 93 can be calculated from intersection point 92 in the same manner. For a given scanline k, the Δxₖ,Δ/k are constant.

This means that once Δₖ (=Δxₖ,Δ/k) is computed for all k then the intersection points for the entire pixel buffer may be computed immediately. These calculations may be performed very quickly and efficiently by hardware vector arithmetic units.

For display purposes, the vector [symbol] ₖ must be sorted in order of increasing xₖ. In order to do this efficiently, sorting is only performed when xₖ₊₁<xₖ for at least one scanline. Where the pixel rows are calculated on a row by row basis, the comparisons and necessary sorting are done as the row is calculated, which is very efficient.

Once the intersection points [symbol] ₖ for a pixel row are established, the brightness value for each pixel is selected as described above.

The result of this repeated processing is an array of values in the pixel grid buffer. These values are brightness values for the related pixel. This array is mapped to the physical pixels of display 18 and the result is a conventional ultrasound image where brightness corresponds to the intensity of echo, compensated for depth attenuation, and a picture of the internal features of the subject is formed.

It can be seen that the method of the invention, incorporating the use of the pixel-scanline co-ordinate system, allows for very efficient operation since most computation is addition.

A further advantage of the method of the invention is that the computation time for producing the pixel buffer array values is almost independent of the spatial density and ordering of the scanline data set. That means that the number of scanlines, the number of data points on each scanline and the manner in which the scanlines are collected has little influence on the computation time. The computation time is primarily dependent on the size of the pixel buffer. This size will be related to the display device employed, and will be fixed or at least known when any device implementing the invention is being designed. This means that a user of the device will see consistent, as designed, image rendering times.

The number of holes, and the remoteness of the valid pixels used to interpolate the values assigned to these holes may be used as a measure of scan quality. This may be calculated and displayed to a user using any convenient scale of quality.

Although the invention has been herein shown and described in what is conceived to be the most practical and preferred embodiment, it is recognised that departures can be made within the scope of the invention, which is not to be limited to the details described herein but is to be accorded the full scope of the appended claims so as to embrace any and all equivalent devices and apparatus.

1. A method for forming an image of a target from echo return data in an ultrasound system including the steps of:
a. moving a probe unit containing an ultrasound transducer over a body to be imaged;
b. receiving an output of a sensor adapted to provide information about the position and/or orientation of the probe unit;
c. receiving echo return data from the ultrasound transducer;
d. combining the sensor output with the echo return data to produce scanlines, each scanline including echo intensity data and information defining the position and/or orientation of the scanline;
e. calculating a transform adapted to map the scanlines to a plane of best fit;
f. applying the transform to the scanlines;
g. mapping the transformed data to a raster image; and
h. displaying the resultant image.

2. The method of claim 1 wherein the calculating of the transform includes principal component analysis.

3. The method of claim 1 wherein the mapping includes using pixel row-wise interpolation.

4. The method of claim 1 wherein the mapping involves producing an array of pixel buffer brightness values, such production including the steps of:
a. determining a first set of intersection points between a centerline of a first pixel row and each scanline;
b. determining a second set of intersection points for a second pixel row adjacent to the first pixel row from the first set of intersection points;
c. assigning the value of a data point on each scanline which is nearest to each intersection to be the pixel buffer brightness value; and
d. where such assignment would result in more than one brightness value for a particular pixel, assigning the average value of the more than one brightness values as the pixel buffer brightness value.

5. The method of claim 4 wherein the intersection points are defined in terms of a co-ordinate system consisting of an index of pixel number along the pixel row and an index of data point number along the scanline.

6. The method of claim 1 wherein the mapping further includes the steps of:
a. overlaying the transformed scanlines with a pixel grid;
b. assigning scanline data values as pixel values to all pixels in which the data values fall;
c. assigning data values to any remaining pixels within an area covered by scanlines by interpolating between previously assigned pixel values;
d. mapping the pixel grid to a display, and
e. displaying an image.

7. The method of claim 2 wherein principal component analysis is applied only to selected data points of a scanline.

8. The method of claim 7 wherein the selected data points are the first and last points of the scanline.

9. The method of claim 1 including the step of calculating and displaying to a user a measure of the quality of the fit of the plane of best fit.

10. The method of claim 9 wherein the quality measure is the variance of a component of co-ordinate values orthogonal to the plane of best fit.

11. The method of claim 3 further including the calculation and display to a user of a measure of scan quality proportional to the degree to which displayed image point values were not directly taken from a scanline value.

12. The method of claim 1 wherein each scanline has direction information for all degrees of freedom.

13. The method of claim 1 wherein each scanline has direction information for at least two but less than all degrees of freedom.

14. The method of claim 1 wherein each scanline has direction information for one degree of freedom.

15. The method of claim 14 wherein the direction information is rotation about a single axis of the probe unit.

16. The method of claim 13 wherein the direction information is rotation about two orthogonal axes of the probe unit.

17. A method of forming an image in an ultrasound system including the steps of:
a. capturing ultrasound scanlines, each scanline including direction and/or position information relative to a first arbitrary co-ordinate system;
b. defining a second co-ordinate system as an optimum co-ordinate system for display of the scanlines in two dimensions;
c. calculating a transform between the first and second co-ordinate systems, applying the transform to the scanlines;
d. overlaying the transformed scanlines with a pixel grid;
e. assigning scanline data values as pixel values to all pixels in which the data values fall;
f. assigning data values to any remaining pixels within an area covered by scanlines by interpolating between previously assigned pixel values;
g. using the pixel values to drive a display screen to form an ultrasound image on the screen.

18. The method of claim 17 wherein the method of assigning scanline data values to pixels further includes the steps of:
a. determining a centerline of a pixel row;
b. determining an intersection between the centerline and any scanline;
c. assigning the value of a data point on the scanline which is nearest to the intersection to be the pixel value.

19. The method of claim 17 wherein a first basis vector of the second frame of reference is chosen as a unit vector in a direction which bisects the angle which is the greatest angle between any two scanlines.

20. The method of claim 19 wherein the steps for calculating the remaining basis vectors of the second frame of reference include:
a. transforming the scanlines into a temporary frame of reference consistent with the first selected basis vector;
b. calculating a covariance matrix for the scanline data;
c. calculating the remaining basis vectors of the second frame of reference as the primary and secondary eigenvectors of the matrix.

21. An apparatus for forming an image in an ultrasound system including:
a. a probe unit having:
(1) at least one transducer adapted to emit and receive a single ultrasound scanline, and
(2) a sensor adapted to sense at least one component of at least one of the probe unit's position and orientation;
b. a data processing and display unit adapted to process and display processed scanline data as an ultrasound image,
wherein in use:
(1) the instantaneous output of the sensor is combined with corresponding transducer output data to form scanline data;
(2) the scanline data is transmitted to the data processing and display unit, wherein the processing of the scanline data includes applying a transform to map the scanline data to a plane of best fit;
(3) mapping and interpolating the transformed data to a raster image, the image being displayed on the data processing and display unit.

22. The apparatus of claim 21 wherein the transform is calculated by principal component analysis.

23. The apparatus of claim 21 wherein the mapping and interpolation is undertaken using pixel row-wise interpolation.

24-26. (canceled)
